(19) United States
(12) Patent Application Publication
(10) Pub. No.: US 2005/0233940 A1
(43) Pub. Date: Oct. 20, 2005

(54) Perfume compositions

(76) Inventors: Keith Douglas Perring, Kent (GB); David Jonathan Bradshaw, Kent (GB); John Martin Behan, Kent (GB); Paula Maria Cawkill, Kent (GB)

Correspondence Address: MORGAN LEWIS & BOCKIUS LLP, 1111 PENNSYLVANIA AVENUE NW, WASHINGTON, DC, 20004, US

(21) Appl. No.: 10/521,432

(22) PCT Filed: Jul. 16, 2003
(86) PCT No.: PCT/GB03/03094

§ 371 Date: Jan. 14, 2005

(30) Foreign Application Priority Data
Jul. 18, 2002 (GB) 0216632.0
Nov. 28, 2002 (GB) 0227742.4

(51) Int. Cl. A61K 7/46

(52) U.S. Cl. 512/1

(57) ABSTRACT

A perfume composition comprising a perfume component capable of inhibiting the production of odoriferous steroids by micro-organisms on the skin. The perfume component is capable of inhibiting bacterial 3a((3)-steroid dehydrogenase and/or steroid 4,5isomerase.

### FIELD OF THE INVENTION

This invention relates to perfume compositions, to products containing such perfume compositions, and to the use of a perfume component or perfume composition to deliver a deodorant effect. In particular, the invention relates to perfume components, mixtures thereof, and perfume compositions, for reducing or preventing body malodour.

### BACKGROUND TO THE INVENTION

It is known that, at the point of secretion, sweat is odourless. Body malodour is the result of a variety of biotransformations of components of sweat by certain species of natural micro-organisms which live on the surface of the skin. These transformations produce a number of volatile odoriferous compounds such as steroidal compounds (e.g. 16-androstenes), amongst others, which contribute to body malodour.

There are three types of personal product routinely used to combat body malodour: perfumes, antiperspirants and deodorants. Products such as soaps, shower gels, body washes and laundry products are also intended to combat body malodour.

Perfumes may simply mask body malodour. However perfume compositions have been disclosed which exhibit a deodorant action. EP-B-3172, EP-A-5618, U.S. Pat. No. 4,304,679, U.S. Pat No. 4,322,308, U.S. Pat. No. 4,278,658, U.S. Pat. No. 4,134,838, U.S. Pat. No. 4,288,341 and U.S. Pat. No. 4,289,641 all describe perfume compositions which exhibit a deodorant action when applied to human skin or when included in a laundry product used to launder textiles.

Antiperspirants work by blocking the sweat glands, thereby reducing perspiration.

Antimicrobial agents used in deodorants are designed to reduce the population, inhibit the growth or diminish the metabolic activities of micro-organisms living on the surface of the skin. Typical agents of this nature include ethanol and Triclosan (2′,4,4′-trichloro-2-hydroxydiphenyl ether) which are well known to exert antimicrobial effects. The use of common deodorant actives results in a non-selective antimicrobial action exerted upon most of the skin's natural microflora. This is an undesirable disadvantage of such deodorant formulations, since the natural microflora provides a protective barrier (colonisation resistance) against invasion by potentially pathogenic bacteria.

U.S. Pat. No. 5,643,559 (Colgate-Palmolive Company) discloses deodorant active materials having an effective amount of Zn²⁺ ions for inhibiting bacterial exoenzymes responsible for the production of axillary malodour. The bacterial exoenzymes are further characterised as aryl sulphatase or beta glucuronidase.

DE-4343265 (Heiakel) describes deodorant compositions comprising saturated dioic acid (C3-C10) esters. The active inhibits a sweat decomposing esterase and the compositions are said not to disturb the skin's natural microflora.

WO 94/07837 (Urichema) describes certain novel unsaturated dioic acids having between 8 and 22 carbon atoms. The potential use of these acids to treat malodour is also described.

Gower et al. (J. Steroid Biochem. Molec. Biol., (1994) Vol. 48, No. 4, pp 409-418) discloses the importance of certain bacterial enzymes involved in bacterial steroid metabolism in the production of odoriferous steroids, and proposes a series of interconversions between some of these metabolites.

Talalay, P.: Hydroxysteroid Dehydrogenases in The Enzymes, VII, 2nd Ed., (Boyer, P., Lardy, H., and Myrback, K., eds.), Academic Press, NY, 177, 1963, describes that 3[[alpha]]hydroxysteroid dehydrogenase from Pseudomonas testosteroni is inhibited by heavy metals and sulfhlydryl-binding reducing agents.

Nakajin et al. (J. Steroid Biochem. Molec. Biol., (1991) January;38(1):95-9) discloses that the—conazole antifungal agents have a mode of action based on the inhibition of steroid metabolism. The activity of the enzyme (16-ene-C19-steroid synthesizing enzyme) responsible for the conversion of C21-steroids to 16-ene-C19-steroids, which was localized on pig testicular microsomes, was inhibited by some typical imidazole antifungal compounds such as clotrimrazole, econazole, miconazole and ketoconazole which are known to be universal inhibitors of cytochrome P-450 dependent enzymes.

Lavallee et al. (J. Steroid Biochem. Molec. Biol. (1993) July;46(1):73-83) describes 20 beta-hydroxypregnenolone as a more potent inhibitor of 5,16-androstadien-3 beta-ol synthetase than of 17-hydroxylase and for the latter enzyme activity, the Ki(app) was lower than that for 17-hydroxypregnenolone itself.

Watabe et al. (J. Biol. Chem. (1985) July 25;260(15):8716-20) describes that the C16-double bond of the steroid androsta-5,16-dien-3 beta-ol, is oxidized by male rat liver microsomes to 16 alpha,17 alpha-epoxyandrost-5-en-3 beta-ol; 16 beta,17 beta-epoxyandrost-5-en-3 beta-ol; androst-5-ene-3 beta,16 alpha,17 beta-triol; and androst-5-ene-3 beta,16 beta, 17 alpha-triol, and this transformation is strongly inhibited with CO.

WO 00/01355 and WO 00101358 describe agents useful in preventing or reducing body malodour by inhibiting the production of odoriferous steroids, wherein the agents inhibit the bacterial enzymes, bacterial 4-ene reductase and/or 5 α-reductase. Examples of active agents are described as dicarboxylic acids, phenyl compounds, monoterpene derivatives, sterols, flavonoids, steryl esters, 2,7-napthalenediol and oxyquinoline (WO 00/01355), and certain perfume components (WO 00/01358).

Several steroids, notably 5α-androst-16-en-3-one (5α-androstenone), 5α-androst-16-en-3α-ol (3α-androstenol) and androsta-4,16-dien-3-one (androstadienone) are known to be highly odorous in the context of human axillary odour. The biotransformations effected by a micro-organism on the components of sweat to produce such odoriferous products or intermediates, occur via a number of possible, and typically, ill-defined metabolic pathways.

### SUMMARY OF THE INVENTION

Bacterial enzymes present in a micro-organism and responsible for the production of odoriferous steroids, particularly the androstadienones such as androstra-5,16-dien-3-one and antrosta-4,16-dien-3-one, include 3α(β)-sterol dehydrogenase and steroid 4,5-isomerase.

Using this knowledge, the present invention is based on extensive testing of perfume components to determine whether a particular component is capable of inhibiting these bacterial enzymes i.e. 3α(β)-sterol dehydrogenase and/or steroid 4,5-isomerase, and thus is capable of unhibiting the production of the odoriferous steroids, e.g. androstadienones, by micro-organisms on the skin surface. Based on this testing, perfume components were identified, which whilst known, possess hitherto unappreciated deodorant properties. The invention thus enables perfume compositions to be defined that reduce or prevent body malodour.

Accordingly, in one aspect, the present invention provides a perfume composition comprising a perfume component capable of inhibiting the production of odoriferous steroids by micro-organisms on the skin characterised in that the perfume component is capable of inhibiting bacterial 3α(β)-sterol dehydrogenase and/or steroid 4,5-isomerase.

Typically, perfume components useful herein are as follows:
N-ethyl-N-(3-methylphenyl)propionamide (also known as ‘Agarbois’ where AGARBOIS is a trade mark of Quest International);
2-ethyl-N-methyl-N-(3-methylphenyl)butanamide (also known as ‘Paradisamide’ where PARADISAMIDE is a trade mark of Quest International);
dihydromyrcenol (2,6-dimethyl-7-octen-2-ol);
(4-isopropylcyclohexyl)methanol;
3-methyl-5-phenylpentan-1-ol (also known as ‘Mefrosol’ where MEFROSOL is a trade mark of Quest International);
2,2,2-trichloro-1-phenylethyl acetate (also known as Rosacetone or Roseacetone);
isobornyl acetate;
allyl amyl glycolate (‘2-methylbutyloxyacetic acid, 2-propenyl ester’);
alpha-terpineol;
acetyl cedrene (also known as ‘Lixetone’ where LIXETONE is a trade mark of Quest International);
tetrahydrogeraniol;
citronellal;
cuminic aldehyde(para-isopropylbenzaldehyde);
cis-jasmone;
pine American oil;
peppermint (Chinese);
1,3,3-trimethyl-2-norbornanol (fenchyl alcohol);
gamma-nonalactone;
octahydro-2H-chromen-2-one (also known as ‘Octahydrocoumarin’ where OCTAHYDROCOUMARIN is a trade mark of Quest International);
cis-4-decenal; and
3-(3-isopropylphenyl)butanal.

Preferably, the perfume component is selected from at least one of the following:
N-ethyl-N-(3-methylphenyl)propionamide;
2-ethyl-N-methyl-N-(3-methylphenyl)butanamide;
(4-isopropylcyclohexyl)methanol;
3-methyl-5-phenylpentan-1-ol;
2,2,2-trichloro-1-phenylethyl acetate;
isobornyl acetate;
allyl amyl glycolate;
acetyl cedrene;
tetrahydrogeraniol;
citronellal;
cuminic aldehyde;
cis-jasmone;
pine American oil;
peppermint (Chinese);
1,3,3-trimethyl-2-norbornanol;
gamma-nonalactone
octahydro-2H-chromen-2-one;
cis-4-decenal; and
3-(3-isopropylphenyl)butanal.

The term “perfume component” is used herein to represent a material Which is added to a perfume composition to contribute to the olfactive properties of the composition. A perfume component can be acceptably employed to provide odour contributions to the overall hedonic performance of products. Typically, a perfume component will be generally recognised as possessing odours in its own right, will be relatively volatile and often has a molecular weight within the range 100 to 300. Typical materials which are perfume components are described in “Perfume and Flavour Chemicals”, Volumes I and II (Steffan Arctander, 1969).

For the purposes of the present invention, by perfume composition is meant a mixture of individual perfume components, and optionally one or more suitable diluents, which is used to impart a desired odour to the skin and/or product for which an agreeable odour is indispensable or desirable. Commonly used diluents are benzyl benzoate, diethyl phthalate, dipropylene glycol and isopropyl myristate. The concentration of perfume components referred to herein is relative to the total concentration of perfume components present in the composition, i.e. excludes any diluents.

Preferably at least 30% by weight of the total weight of the perfume composition is comprised of one or more perfume components capable of inhibiting 3α(β)-sterol dehydrogenase and/or steroid 4,5-isomerase.

Thus, to deliver high deodorant effects the perfume component(s) preferably comprise(s) at least 30% by weight of the total weight of a perfume composition, more preferably at least 40%, even more preferably at least 45% and most preferably at least 60%.

In a further aspect, the invention provides a perfume composition comprising at least 30% by weight of one or more of the following perfume components: N-ethyl-N-(3-methylphenyl)propionamide; 2-ethyl-N-methyl-N-(3-methylphenyl)butanamide; dihydromyrcenol; (4-isopropylcyclohexyl)methanol; 3-methyl-5-phenylpentan-1-ol; 2,2,2-trichloro-1-phenylethyl acetate; isobornyl acetate; allyl amyl glycolate; alpha-terpineol; acetyl cedrene; tetrahydrogeraniol; citronellal; cuminic aldehyde; cis-jasmone; pine American oil; peppermint (Chinese); 1,3,3-trimethyl-2-norbornanol; gamma-nonalactone; octahydro-2H-chromen-2-one; cis-4-decenal; 3-(3-isopropylphenyl)butanal.

Additionally or alternatively, a perfume composition preferably comprises at least 3, more preferably at least 5, and even more preferably at least 10 of the specified perfume components.

Thus, in an even further aspect, the present invention provides a perfume composition comprising at least 3 of the following perfume components: N-ethyl-N-(3-methylphenyl)propionamide; 2-ethyl-N-methyl-N-(3-methylphenyl)butanamide; dihydromyrcenol; (4-isopropylcyclohexyl)methanol; 3-methyl-5-phenylpentan-1-ol; 2,2,2-trichloro-1-phenylethyl acetate; isobornyl acetate; allyl amyl glycolate; alpha-terpineol; acetyl cedrene; tetrahydrogeraniol; citronellal; cuminic aldehyde; cis-jasmone; pine American oil; peppermint (Chinese); 1,3,3-trimethyl-2-norbornanol; gamma-nonalactone; octahydro-2H-chromen-2-one; cis-4-decenal; 3-(3-isopropylphenyl)butanal.

The perfume components useful herein in a perfume composition may be incorporated into deodorant products which include, but are not limited to, body deodorants and antiperspirants including roll ons, sprays, gel products, stick deodorants, antiperspirants, shampoos, soaps, shower gels, talcum powder, hand creams, skin conditioners, sunscreens, sun tan lotions, and hair conditioners.

Thus, in a further aspect, the present invention provides a deodorant product comprising a perfume composition in accordance with the invention.

A deodorant product preferably comprises at least 0.05% to 4%, more preferably 0.1% to 2% of a perfume composition by weight of the deodorant product.

The perfume components useful herein may also be conveniently employed for deodorant purposes by incorporation into other products, e.g. laundry and household products such as rinse conditioners, household cleaners and detergent cleaners. The perfume components can be incorporated into textiles themselves during their production using techniques known in the art, to provide deodorant protection.

In a preferred embodiment of the present invention, an Odour Reduction Value, measured in human axillae as described in Example 4, of at least 10%, more preferably at least 30%, and particularly at least 45% is obtained.

One or more of the perfume components useful herein may be mixed with other perfume components, e.g. perfume components of the prior art having deodorant properties, to formulate perfume compositions with desired deodorant and hedonistic properties.

In one such embodiment, there is provided a perfume composition as defined herein, wherein the perfume composition additionally comprises at least 15% by weight, preferably at least 30% by weight, of the following perfume components: acetyl di-iso-amylene, acetyl tributyl citrate, aldehyde C10 (i.e. decenal), Amber AB 358 (available from Quest International), amyl salicylate, anisyl acetate, Azarbre*, benzyl salicylate, cis-3-hexenyl salicylate, citral, citronellol, clove leaf distilled, coriander, cyclamen aldehyde, decen-1-ol, dihydroeugenol, diphenylmethane, Dupical*, Empetaal*, geraniol, helional i.e. 2-methyl-3-(3,4-methylene-dioxyphenyl)propanal), ionones (alpha- and beta-), Jasmacyclene*, 3-(4-methyl-4-hydroxyamyl)-3-cyclohexene carboxaldehyde, methyl eugenol, methyl isoeugenol, Ortholate*, para-cresyl methyl ether, 2-phenylethyl alcohol, para tert. butyl cyclohexyl acetate, rose oxide (racemic), styrallyl acetate, tetrahydrolinalol, and vanillin; wherein all asterisked materials are trade marks of Quest International.

In a preferred embodiment, there is provided a perfume composition comprising:
(i) at least 30% by weight of the perfume composition of at least 3 of the following perfume components: N-ethyl-N-(3-methylphenyl)propionamide, 2-ethyl-N-methyl-N-(3-methylphenyl)butanamide, dihydromyrcenol, (4-isopropylcyclohexyl)methanol, 3-methyl-5-phenylpentan-1-ol, 2,2,2-trichloro-1-phenylethyl acetate, isobornyl acetate, allyl amyl glycolate, alpha-terpineol, acetyl cedrene, tetrahydrogeraniol, citronellal, cuminic aldehyde, cis-jasmone, pine American oil, peppermint (Chinese), 1,3,3-trimethyl-2-norbornanol, gamma-nonalactone, octahydro-2H-chromen-2-one, cis-4-decenal, 3-(3-isopropylphenyl)butanal; and
(ii) at least 30% by weight of the perfume composition of one or more of the following perfume components: acetyl di-iso-amylene, acetyl tributyl citrate, aldehyde C10, Amber AB 358, amyl salicylate, anisyl acetate, Azarbre, benzyl salicylate, cis-3-hexenyl salicylate, citral, citronellol, clove leaf distilled, coriander, cyclamen aldehyde, decen-1-ol, dihydroeugenol, diphenylmethane, Dupical, Empetaal, geraniol, helional, alpha-ionone, beta-ionone, Jasmacyclene, 3-(4-methyl-4-hydroxyamyl)-3-cyclohexene carboxaldehyde, methyl eugenol, methyl isoeugenol, Ortholate, para-cresyl methyl ether, 2-phenylethyl alcohol, para tert. butyl cyclohexyl acetate, rose oxide, styrallyl acetate, tetrahydrolinalol, and vanllin.

Also included within the scope of the invention is a method, particularly a cosmetic method, for reducing or preventing body malodour by topically applying to human skin a composition comprising a perfume component selected from at least one of the following:
N-ethyl-N-(3-methylphenyl)propionamide; 2-ethyl-N-methyl-N-(3-methylphenyl)butanamide; dihydromyrcenol; (4-isopropylcyclohexyl)methanol; 3-methyl-5-phenylpentan-1-ol; 2,2,2-trichloro-1-phenylethyl acetate; isobornyl acetate; allyl amyl glycolate; alpha-terpineol; acetyl cedrene; tetrahydrogeraniol; citronellal; cuminic aldehyde; cis-jasmone; pine American oil; peppermint (Chinese); 1,3,3-trimethyl-2-norbornanol; gamma-nonalactone; octahydro-2H-chromen-2-one; cis-4-decenal; 3-(3-isopropylphenyl)butanal.

Preferably, the composition is a perfume composition.

The method thus comprises topically applying to human skin, one or more of the specified perfume components which is(are) capable of reducing or preventing body malodour by inhibiting the production of odoriferous steroids by micro-organisms present on the skin surface, wherein the perfume component is capable of inhibiting the bacterial enzyme(s), 3α(β)sterol-dehydrogenase and/or steroid 4,5-isomerase. Typically, the specified perfume components inhibit the production of odoriferous steroids by Coryneform bacteria present on the skin surface, particularly Corynebacterium spp. The inhibitory effect of the perfume components useful herein can be achieved antimicrobially or sub-lethally.

The antimicrobial effects of compounds, e.g. perfume components, are usually divided into two types; they can either inhibit bacterial growth (bacteriostatic action) or alternatively they can act by directly killing existing viable bacteria (bactericidal action).

The bacteriostatic action of a compound “X” such as a perfume component, can be tested for in vitro by inoculating a standard, small number of bacteria into broths containing an appropriate range of concentrations of X. The broths are then incubated for a suitable time, and growth compared with a control containing no inhibitor. The broth containing the lowest concentration of X which shows reduction of growth compared to the control broth is defined as the minimum inhibitory concentration (MIC).

The determination of bactericidal action of a compound “Y” such as a perfume component is carried out by adding various concentrations of compound Y to replicate broths containing relatively high, standard numbers of bacteria. After a certain period allowing any antibacterial activity to take place, aliquots of the bacterial cultures are diluted (usually in 10-fold steps) and dispensed onto agar plates. The plates are incubated with the expectation that each viable cell should produce a visible colony. The numbers of colonies are multiplied to take account of the dilution, to establish the number of viable cells in the broths. Once again, the broths containing compound Y are compared with an untreated control broth. The minimum concentration of compound Y which causes a reduction in the viable number of bacteria is the minimum bactericidal concentration (NMC). NBC can also be expressed in terms of the MBC required to produce a certain degree of killing (for example, a 3 log₁₀ reduction in count, equivalent to a 99.9% kill). Still further, the MBC can be expressed in kinetic terms—the time of exposure to an agent required for a given MBC effect.

A further possibility is that the process of inhibition could be sub-lethal (or sub-SHC), whereby the perfume components interfere with the metabolic process, but typically do not inhibit bacterial growth.

Preferably, the bacterial production of odoriferous steroids is reduced by at least 50%, more preferably by at least 70%, particularly by at least 80%, and especially by at least 90%. Three modes of achieving a reduction of odoriferous steroid production are possible. In the first mode, the perfume components (or perfume compositions) may act by direct (overt antimicrobial) killing of skin bacteria, e.g. by more than 10-fold; in the second mode, they may act on odoriferous steroid generation whilst maintaining a microbial cell viability of at least 70%; in the third mode, they may inhibit odoriferous steroid generation, at a concentration below the minimum inhibitory concentration (MIC), determined as described in Example 1 below. The third mode is preferred, since this provides malodour counteraction benefits, whilst leaving the natural skin microflora undisturbed. Thus, preferably the bacterial production of odoriferous steroids can be reduced or eliminated without significantly disturbing the skin's natural microflora. This may be achieved by sub-lethally inhibiting the bacterial enzymes 3α(β)-sterol dehydrogenase and/or steroid 4,5-isomerase responsible for the production of odoriferous steroids such as the androstadienones, e.g. androsta-5,16-dien-3-one and androsta-4,16-dien-3-one.

In an even further aspect, the present invention provides use of one or more of the following perfume components: N-ethyl-N-(3-methylphenyl)propionamide; 2-ethyl-N-methyl-N-(3-methylphenyl)butanamide; (4-isopropylcyclohexyl)methanol; 3-methyl-5-phenylpentan-1-ol; 2,2,2-trichloro-1-phenylethyl acetate; isobornyl acetate; allyl amyl glycolate; acetyl cedrene; tetrahydrogeraniol; citronellal; cuminic aldehyde; cis-jasmone; pine American oil; peppermint (Chinese); 1,3,3-trimethyl-2-norbornanol; gamma-nonalactone; octahydro-2H-chromen-2-one; cis-4-decenal; 3-(3-isopropylphenyl)butanal; as a deodorant active.

The invention also provides use of one or more of the following perfume components: N-ethyl-N-(3-methylphenyl)propionamide; 2-ethyl-N-methyl-N-(3-methylphenyl)butanamide; (4-isopropylcyclohexyl)methanol; 3-methyl-5-phenylpentan-1-ol; 2,2,2-trichloro-1-phenylethyl acetate; isobornyl acetate; allyl amyl glycolate; acetyl cedrene; tetrahydrogeraniol; citronellal; cuminic aldehyde; cis-jasmone; pine American oil; peppermint (Chinese); 1,3,3-trimethyl-2-norbornanol; gamma-nonalactone; octahydro-2H-chromen-2-one; cis-4-decenal; 3-(3-isopropylphenyl)butanal, in the manufacture of a composition for reducing or preventing body malodour.

The invention also provides the use of a perfume component to reduce body malodour characterised in that the perfume component is capable of inhibiting 3α(β)-sterol dehydrogenase and/or steroid 4,5-isomerase.

The invention further provides the use of a perfume composition, comprising at least 30% by weight of one or more perfume components capable of inhibiting 3α(β)-sterol dehydrogenase and/or steroid 4,5-isomerase, to reduce body malodour.

The invention further provides a method for reducing or preventing body malodour by topically applying to human skin a composition comprising a perfume component which inhibits 3α(β)-sterol dehydrogenase and/or steroid 4,5-isomerase.

The invention still further provides a method of producing a perfume composition which comprises (i) evaluating perfume components on the ability to inhibit 3α(β)-sterol dehydrogenase and/or steroid 4,5-isomerase, (ii) selecting perfume components on the ability to inhibit 3α(β)-sterol dehydrogenase and/or steroid 4,5-isomerase, and (ii) mixing together two or more of said selected perfume components, optionally with other perfume components.

The invention still further provides use of a perfume composition comprising a perfume component to reduce body malodour, characterised in that the composition comprises at least 30% by weight of at least one of the perfume components specified in the paragraph bridging pages 4 and 5 above.

The invention is illustrated by the following examples.

### EXAMPLE 1

### Minimum Inhibitory Concentration (MIC)

The minimum inhibitory concentration of a perfume component was determined by the following method.

A culture of the test strain—Corynebacterium xerosis NCTC 7243 (National Collection of Type Cultures, Public Health Laboratory Service, Central Public Health Laboratory, 61 Colindale Avenue, London, NW9 5HT) was grown in 100 ml of tryptone soya broth (TSB) (Oxoid, Basingstoke, UK) for 16-24 hours, in a shaken flask at 37° C. The culture was then diluted in sterile 0.1% TSB (Oxoid, Basingstoke, UK) to give a concentration of bacteria of approximately 10⁶ colony forming units (cfu) per ml.

Perfume or perfume component samples were diluted in sterile TSB to give stock solutions with final concentrations of 40,000 ppm (perfume) or 20,000 ppm (perfume component). Each row of a standard, 96-well plastic microtitre plate (labelled A-H) was allocated to one sample, thus eight samples per plate. Row H contained only TSB for use as a bacterial control to indicate the degree of turbidity resulting from bacterial growth in the absence of any test material. Aseptically, 200 μl of the initial dilution of perfume/perfume component was transferred to the 1^{st} and 7^{th} well of the appropriate row. All other test wells were filled with 100 μl of sterile TSB using an 8-channel micro-pipette. The contents of each of the wells in column 1 were mixed by sucking samples up and down in pipette tips, before 100 μl was transferred to column 2. The same sterile pipette tips were used to transfer 100 μl of each well in column 7, into the appropriate well in column 8. This set of eight tips was then discarded into disinfectant solution. Using eight fresh, sterile tips the process was repeated by transferring 100 μl from column 2 into column 3 (and 8 into 9). The process was continued until all wells in columns 6 and 12 contained 200 μl. After mixing, 100 μl was discarded from wells in columns 6 and 12 to waste. Finally, 100 μl of pre-diluted bacterial culture (approx. 10⁶ cfu/ml) was added, thus giving 200 μl final volume in each well.

A blank plate was prepared for each set of eight samples in exactly the same way, except that 100 μl of sterile 0.1% TSB was added instead of bacterial culture. This plate was used as the control plate against which the test plate(s) could be read. Test and control plates were sealed using autoclave tape and incubated for 18-30 hours at 37° C.

A microtitre plate reader (Model MRX, Dynatech Laboratories) was preset to gently agitate the plates and to mix the contents. The absorbance at 540 nm (hereinafter referred to for brevity and simplicity as “A₅₄₀”) was used as a measure of turbidity resulting from bacterial growth. The control, un-inoculated plate for each set of samples was read first, and the plate reader then programmed to use the control readings to blank all other plate readings for the inoculated plates for the same set of test materials (i.e. removing turbidity due to perfume and possible colour changes during incubation). Thus, the corrected readings generated were absorbances resulting from turbidity from bacterial growth. The MIC was taken as the concentration of perfume/perfume component required to inhibit growth so that the change in absorbance during the incubation period was <0.2 A₅₄₀.

### EXAMPLE 2

### Steroid Biotransformation Assay

The ability of perfume components and mixtures of these components to inhibit the bacterial enzymes, 3α(β)-sterol dehydrogenase and/or steroid 4,5-isomerase was determined in vitro using the method described below.

Corynebacterium sp. NCIMB 41018 (National Collections Of Industrial, Food and Marine Bacteria, 23 St Machar Drive, Aberdeen, AB24 3RY, Scotland, UK) (also known as Corynebacterium G41) was grown in 100 ml of TSB supplemented with 0.1% w/v yeast extract (Oxoid) and 0.1% v/v Tween 80 (Sigma, Poole, UK) for 18-30 hours, in a shaken flask at 37° C. This culture was then harvested by centrifugation, and resuspended in 100 ml of biotransformation medium (consisting of a sterile semi-synthetic basal medium containing KH₂PO₄ 1.6 g/l; (NH₄)₂HPO₄ 5 g/l; Na₂SO₄ 0.38 g/l; yeast nitrogen base 3.35 g/l; yeast extract 0.5 g/l; Tween 80 0.2 g/l; Triton X-100 0.2 g/l and MgCl₂.6H₂O 0.5 g/l).

Substrate androsta-5,16-dien-3β-ol (50 mg/assay) was added to the bacterial suspension and incubated for 72 hours at 37° C. with agitation (at 220-250 rpm) in a 250 ml, baffled-Erlenmeyer flask.

Following biotransformation of androsta-5,16-dien-3β-ol to androsta-4,16-dien-3-one the bacteria were harvested and the cell pellet dried in air and then under vacuum.

The dried cells were then crushed and suspended in 100 ml of a mixture of diethyl ether, chloroform, ethanol, ethyl acetate and acetone (1:2:1:1:1 v/v, respectively), and stirred for 16 hours. The supernatant was then reduced to half its volume, filtered and evaporated at 30° C. and 15 mmHg pressure. The resulting residue was re-dissolved in 5 ml AR grade methanol. Following sonication, the sample was analysed by HPLC on a Phenomenex Luna 5 micron, C18 reverse-phase HPLC column coupled to a Millipore-Waters 600E System Controller. Elute was passed through a Millipore-Waters 486 Tuneable absorbance detector and relative amounts of the steroid metabolite was determined by a Hewlett Packard HP 3396A Integrator printer. The composition of the HPLC mobile phase was aqueous methanol. The flow rate was 0.8 mil/min. Calibration curves were used to determine the molar quantities of pure steroid metabolites in biotransformed mixtures and hence the conversions.

Metabolites were analysed by HPLC-MS to determine their structure.

Without wishing to be unduly bound or limited by theory, based upon our results, we propose the following scheme of biotransformations of steroids by Corynebacterium NCIMB 41018.

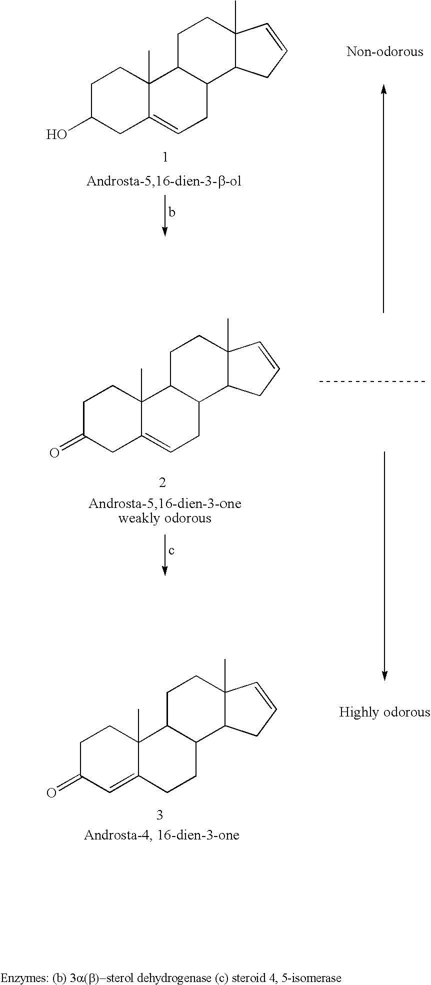

It will be appreciated that the Coryneform bacteria used in Examples 1 and 2 are not the same strains. This is because the nutrient Tween-80 required for growth by Corynebacterium NCIMB 41018 (Example 2) is not suitable for inclusion in the growth medium used for MIC testing. As described above, during MIC testing, measurements are taken of the turbidity resulting from bacterial growth. Tween-80 when dissolved in an aqueous growth medium turns the medium cloudy. Thus, the addition of Tween-80 to a growth medium to be used for MIC testing would interfere with the readings, making an accurate determination of the turbidity due to bacterial growth impossible. Thus, a similar axillary Corynebacterium strain (C. xerosis NCTC 7243) is used in the MIC test, which does not require this nutrient for growth. The susceptibility of Corynebacterium xerosis NCTC 7243 to a variety of perfume components is likely to be very similar to that of Corynebacterium NCIMB 41018 as they are from the same genus.

### EXAMPLE 3

| INGREDIENT | w/w % | |
|---|---|---|
| Perfume A: Composition % by weight. | | |
| AGARBOIS (Q) | 15 | * |
| CINNAMIC ALCOHOL | 2 | |
| COUMARIN | 1 | |
| DIHYDROMYRCENOL | 8 | * |
| GERANIUM OIL | 2 | |
| HABANOLIDE (F) | 3 | |
| LILIAL (G) | 10 | |
| (4-ISOPROPYLCYCLOHEXYL)METHANOL | 2 | * |
| MEFROSOL (Q) | 5 | * |
| METHYL ANTHRANILATE | 1 | |
| METHYL CEDRYL KETONE | 4 | |
| METHYL DIHYDROJASMONATE (Q) | 10 | |
| PHENYL ETHYL ALCOHOL | 15 | |
| ROSACETONE | 5 | * |
| VANILLIN 5% IN DEP | 17 | |
| total | 100.00% | |
| Perfume B: Composition % by weight. | | |
| ACETYL CEDRENE | 7.5 | * |
| AGARBOIS (Q) | 6 | * |
| ALDEHYDE MNA 10% DEP | 1 | |
| ALLYL AMYL GLYCOLATE (Q) | 2.2 | * |
| AMBER CORE (Q) | 0.5 | |
| ARMOISE TUNISIAN | 0.4 | |
| BANGALOL (Q) | 0.5 | |
| BENZYL SALICYLATE (Q) | 8.5 | |
| BERGAMOT OIL | 7.5 | |
| BOURGEONAL (Q) | 0.5 | |
| CARVONE LAEVO (Q) 10% DEP | 1 | |
| CEDARWOOD VIRGINIAN OIL | 1.1 | |
| cis-3-HEXENYL SALICYLATE | 1.5 | |
| CISTULATE (Q) 10% DEP | 2 | |
| CORIANDER | 0.3 | |
| COUMARIN | 0.6 | |
| CYCLOHEXYLOXYACETIC ACID, | 0.2 | |
| ALLYL ESTER | | |
| CYCLOPENTADECANOLIDE | 2.2 | |
| DIHYDROMYRCENOL (Q) | 13 | * |
| ETHYLENE BRASSYLATE | 1.5 | |
| GERANIUM OIL | 1.4 | |
| HELIONAL | 0.3 | |
| HEXYL CINNAMIC ALDEHYDE | 2.5 | |
| IONONE (Q) | 1.5 | |
| ISO AMBOIS (Q) | 7.5 | |
| ISO BORNYL ACETATE | 0.6 | * |
| ISOBORNYL CYCLOHEXANOL | 1.5 | |
| LAVANDIN OIL | 0.3 | |
| LILIAL (G) | 6.8 | |
| METHYL CHAVICOL | 1.2 | |
| METHYL DIHYDROJASMONATE | 6.4 | |
| SUPER (Q) | | |
| MOSS OAKMOSS SYNTHETIC | 0.2 | |
| NUTMEG PURE | 0.2 | |
| PEPPERMINT CHINESE 10% DEP | 3.5 | * |
| PETITGRAIN PARAGUAY | 0.2 | |
| ROSE OXIDE RACEMIC 10% DEP | 0.5 | |
| STYRALLYL ACETATE | 0.4 | |
| TERPINEOL ALPHA | 2.5 * | |
| TETRAHYDROLINALOL | 4.5 | |
| total | 100.00% | |
| Perfume C: Composition % by weight. | | |
| ACETYL CEDRENE (Q) | 7 | * |
| AGARBOIS (Q) | 15 | * |
| ALDEHYDE MNA 10% DEP | 2.5 | |
| BENZYL SALICYLATE (Q) | 6.4 | |
| cis-JASMONE | 1.2 | * |
| CITRONELLAL | 2.2 | * |
| COUMARIN | 1.3 | |
| CYCLOPENTADECANOLIDE | 6.6 | |
| DEHYDROMYRCENOL (Q) | 8.5 | * |
| ETHYLENE BRASSYLATE | 2.3 | |
| HEXYL CINNAMIC ALDEHYDE | 3.5 | |
| ISO AMBOIS (Q) | 7 | |
| ISO BORNYL ACETATE | 2.6 | * |
| LILIAL (G) | 5.4 | |
| MARENIL (Q) | 1.3 | |
| MEFROSOL (Q) | 5.4 | * |
| METHYL DIHYDROJASMONATE | 7.6 | |
| SUPER (Q) | | |
| PETITGRAIN PARAGUAY | 1.2 | |
| TERPINEOL ALPHA | 3 | * |
| TETRAHYDROGERANIOL | 10 | * |
| total | 100.00% | |
| Perfume D: Composition % by weight. | | |
| 4-(5-ETHYLBICYCLO[2.2.1]HEPTYL-2)- | 1.2 | |
| CYCLOHEXANOL | | |
| ACETYL CEDRENE (Q) | 5.3 | * |
| ALDEHYDE C11 (UNDECYLENIC | 1.4 | |
| ALDEHYDE) 10% DEP | | |
| ALDEHYDE MNA 10% DEP | 0.8 | |
| ALLYL AMYL GLYCOLATE (Q) | 1.3 | * |
| ARMOISE TUNISIAN | 0.2 | |
| BANGALOL (Q) | 0.3 | |
| BENZYL SALICYLATE (Q) | 5.1 | |
| BERGAMOT OIL | 4.8 | |
| CEDARWOOD VIRGINIAN OIL | 1.1 | |
| CITRONELLAL | 2 | * |
| CITRONELLOL | 6.9 | |
| CYCLOPENTADECANOLIDE | 2.3 | |
| DIHYDROMYRCENOL (Q) | 15.8 | * |
| ETHYLENE BRASSYLATE | 8.8 | |
| FENCHYL ACETATE | 2.5 | |
| HEXYL CINNAMIC ALDEHYDE | 5.1 | |
| IONONE (Q) | 3.5 | |
| ISOBORNYL CYCLOHEXANOL | 1.8 | |
| METHYL DIHYDROJASMONATE | 5.5 | |
| SUPER (Q) | | |
| PARA TERT BUTYL | 3.4 | |
| CYCLOHEXYL ACETATE | | |
| PARADISAMIDE (Q) | 2.8 | * |
| PEPPERMINT CHINESE 10% DEP | 4.3 | * |
| PHENYLETHYL ALCOHOL | 6 | |
| ROSE OXIDE RACEMIC 10% DEP | 2.1 | |
| ROSEACETONE | 3.7 | * |
| TETRAHYDROGERANIOL | 2 | * |
| total | 100.00% | |
| Perfume E: Composition % by weight. | | |
| 4-(5-ETHYLBICYCLO[2.2.1]HEPTYL-2)- | 2.3 | |
| CYCLOHEXANOL | | |
| AGARBOIS (Q) | 4 | * |
| ALDEHYDE C11 (UNDECYLENIC ALDEHYDE) | 1.2 | |
| 10% DEP | | |
| AMBER CORE (Q) | 4.3 | |
| CARVONE LAEVO (Q) 10% DEP | 3.8 | |
| CEDARWOOD VIRGINIAN OIL | 1.8 | |
| cis-JASMONE | 0.5 | * |
| CISTULATE (Q) 10% DEP | 0.9 | |
| CITRONELLOL | 3.6 | |
| CORIANDER | 0.2 | |
| COUMARIN | 0.9 | |
| DIHYDROMYRCENOL (Q) | 4.5 | * |
| ETHYLENE BRASSYLATE | 6.2 | |
| FENCHYL ACETATE | 3.6 | |
| HEXYL CINNAMIC ALDEHYDE | 6.8 | |
| HEXYL SALICYLATE | 7.5 | |
| LILIAL (G) | 6.5 | |
| MARENIL (Q) | 2.6 | |
| METHYL CHAVICOL | 0.4 | |
| METHYL DIHYDROJASMONATE | 3.5 | |
| SUPER (Q) | | |
| METHYL OCTYL ACETALDEHYDE | 5.5 | |
| 10% DEP | | |
| MOSS OAKMOSS SYNTHETIC | 0.2 | |
| PEPPERMINT CHINESE 10% DEP | 3.4 | * |
| PETITGRAIN PARAGUAY | 2.1 | |
| PHENYLETHYL ALCOHOL | 7.1 | |
| TERPINEOL ALPHA | 6.4 | * |
| TETRAHYDROGERANIOL | 8.2 | * |
| TETRAHYDROLINALOL | 2 | |
| total | 100.00% | |

* Materials of the invention
Trademarks: ‘Q’ = Quest International; ‘F’ = Firmenich; ‘G’ = Givaudan

### EXAMPLE 4

### Product Base Examples

The following are typical formulations of deodorant products which comprise a perfume composition in accordance with the invention. These formulations are made by methods common in the art.

1. Deodorant Sticks

Content (% by weight)

| Ingredient | Formulation 1A | Formulation 1B |
|---|---|---|
| Ethanol | | 8.0 |
| Sodium Stearate | 7.0 | 6.0 |
| Propylene glycol | 70.0 | 12.0 |
| Perfume | 1.5 | 2.0 |
| PPG-3 Myristyl | | 28.0 |
| ether | | |
| PPG-10 Cetyl ether | | 10.0 |
| Cyclomethicone | | 34.0 |
| Water | 21.5 | |

2. Aerosols

Content % by weight

| Ingredient | Formulation 2A | Formulation 2B |
|---|---|---|
| Ethanol B | up to 100 | |
| Propylene glycol | as required | |
| Perfume | 2.0 | 1.2 |
| Chlorhydrol microdry | | 31.8 |
| Silicone Fluid DC344 | | up to 100 |
| Bentone gel IPP | | 13.65 |
| Dimethyl ether | 20.0 | |
| Concentrate | | 22.0 |
| Water | 23.0 | |

| | Content % by weight |
|---|---|
| Ingredient | Formulation 2C |
| Ethanol (Denatured) | up to 100 |
| Perfume | 1.0 |
| DC345 Fluid ® | 15.0 |
| Hydrocarbon Propellant, 30 psig⁽ⁱⁱ⁾ | 60.0 |

3. Roll ons

Content % by weight

| Ingredient | Formulation 3A | Formulation 3B |
|---|---|---|
| Ethanol | to 100% | 60.0 |
| Klucel MF | | 0.65 |
| Cremphor RM410 | | 0.5 |
| Perfume | 0.5 | 1.0 |
| AZTC* | 20.0 | |
| Cyclomethicone | 68.0 | |
| Dimethicone | 5.0 | |
| Silica | 2.5 | |
| Water | | 37.85 |

* Aluminium zirconium tetrachlorohydro glycinate
⁽ⁱ⁾DC345 fluid (INCI name - CYCLOPENTA-SELOXANE) is a volatile, low
viscosity, silicone fluid. It is non-greasy providing a light, silky feel on the skin.
⁽ⁱⁱ⁾The hydrocarbon propellant can be any deodorised blend of n-butane, n-propane and isobutane having a pressure of 30 pounds per square inch gauge or 2.109 kg/cm² gauge (308 kPa).

Perfume compositions A to E embodying this invention (see Example 3 above) were made and tested for deodorant action in underarm products, particularly an aerosol product of

Formulation 2C, using an Odour Reduction Value test generally as described in U.S. Pat. No. 4,278,658.

The Odour Reduction Value test was carried out using a panel of 40 Caucasian male subjects. A standard quantity (approximately 1.75 g) of an aerosol product containing one of the perfume compositions or an unperfumed control was applied to the axillae of the panel members in accordance with a statistical design.

After a period of five hours, the underarm odour was judged by three trained female assessors who scored the odour intensity in accordance with a 0 to 5 scale, as shown below:

| | | Conc, of aqueous |
|---|---|---|
| Score | Odour level | isovaleric acid (ml/I) |
| 0 | No odour | 0 |
| 1 | Slight | 0.013 |
| 2 | Definite | 0.053 |
| 3 | Moderate | 0.22 |
| 4 | Strong | 0.87 |
| 5 | Very Strong | 3.57 |

Average scores for each test product and the control product were then determined. The score for each test product was subtracted from the score for the control product and the reduction expressed as a percentage to give the Odour Reduction Value(%).

Perfume compositions A to E were all found to exhibit significant deodorant activity.

For example, Perfume A contains 35% of perfume components of the invention. Excluding diluents, this percentage increases to 42.2%. For this perfume, present at 1.0% in an aerosol product of Formulation 2C above, the Odour Reduction Value(%) compared to an unperfumed control was 48.3% (5 hours).

The Odour Reduction Value (%) compared to an unperfamed control for Perfume B was 44.6% (5 hours), for Perfume C 35.3% (5 hours) and for Perfumne E 28.2% (5 hours).

1. A perfume composition comprising a perfume component capable of inhibiting the production of odoriferous steroids by micro-organisms on the skin characterised in that the perfume component is capable of inhibiting bacterial 3α(β)-sterol dehydrogenase and/or steroid 4,5-isomerase.

2. A perfume composition according to claim 1, wherein the composition comprises at least 30% by weight of at least one perfume component capable of inhibiting bacterial 3α(β)-sterol dehydrogenase and/or steroid 4,5-isomerase.

3. A perfume composition comprising at least 30% by weight of one or more of the following perfume components: N-ethyl-N-(3-methylphenyl)propionamide; 2-ethyl-N-methyl-N-(3-methylphenyl)butanamide; dihydromyrcenol; (4-isopropylcyclohexyl)methanol; 3-methyl-5-phenylpentan-1-ol; 2,2,2-trichloro-1-phenylethyl acetate; isobornyl acetate; allyl amyl glycolate; alpha-terpineol; acetyl cedrene; tetrahydrogeraniol; citronellal; cuminic aldehyde; cis-jasmone; pine American oil; peppermint (Chinese); 1,3,3-trimethyl-2-norbornanol; gamma-nonalactone; octahydro-2H-chromen-2-one; cis-4-decenal; 3-(3-isopropylphenyl)butanal.

4. A perfume composition comprising at least 3 of the following perfume components: N-ethyl-N-(3-methylphenyl)propionamide; 2-ethyl-N-methyl-N-(3-methylphenyl)butanamide; dihydromyrcenol; (4-isopropylcyclohexyl)methanol; 3-methyl-5-phenylpentan-1-ol; 2,2,2-trichloro-1-phenylethyl acetate; isobornyl acetate; allyl amyl glycolate; alpha-terpineol; acetyl cedrene; tetrahydrogeraniol; citronellal; cuminic aldehyde; cis-jasmone; pine American oil; peppermint (Chinese); 1,3,3-trimethyl-2-norbornanol; gamma-nonalactone; octahydro-2H-chromen-2-one; cis-4-decenal; 3-(3-isopropylphenyl)butanal.

5. A perfume composition according to claim 4, wherein the perfume composition comprises at least 30% by weight of at least 3 of the specified perfume components.

6. A deodorant product comprising a perfume composition according to any one of the preceding claims.

7. Use of one or more of the following perfume components: N-ethyl-N-(3-methylphenyl)propionamide; 2-ethyl-N-methyl-N-(3-methylphenyl)butanamide; (4-isopropylcyclohexyl)methanol; 3-methyl-5-phenylpentan-1-ol; 2,2,2-trichloro-1-phenylethyl acetate; isobornyl acetate; allyl amyl glycolate; acetyl cedrene; tetrahydrogeraniol; citronellal; cuminic aldehyde; cis-jasmone; pine American oil; peppermint (Chinese); 1,3,3-trimethyl-2-norbornanol; gamma-nonalactone; octahydro-2H-chromen-2-one; cis-4-decenal; 3-(3-isopropylphenyl)butanal; as a deodorant active.

8. Use of one or more of the following perfume components: N-ethyl-N-(3-methylphenyl)propionamide; 2-ethyl-N-methyl-N-(3-methylphenyl)butanamide; (4-isopropylcyclohexyl)methanol; 3-methyl-5-phenylpentan-1-ol; 2,2,2-trichloro-1-phenylethyl acetate; isobornyl acetate; allyl amyl glycolate; acetyl cedrene; tetrahydrogeraniol; citronellal; cuminic aldehyde; cis-jasmone; pine American oil; peppermint (Chinese); 1,3,3-trimethyl-2-norbornanol; gamma-nonalactone; octahydro-2H-chromen-2-one; cis-4-decenal; 3-(3-isopropylphenyl)butanal, in the manufacture of a composition for reducing or preventing body malodour.

9. A method for reducing or preventing body malodour by topically applying to human skin a composition comprising a perfume component selected from at least one of the following: N-ethyl-N-(3-methylphenyl)propionamide; 2-ethyl-N-methyl-N-(3-methylphenyl)butanamide; dihydromyrcenol; (4-isopropylcyclohexyl)methanol; 3-methyl-5-phenylpentan-1-ol; 2,2,2-trichloro-1-phenylethyl acetate; isobornyl acetate; allyl amyl glycolate; alpha-terpineol; acetyl cedrene; tetrahydrogeraniol; citronellal; cuminic aldehyde; cis-jasmone; pine American oil; peppermint (Chinese); 1,3,3-trimethyl-2-norbornanol; gamma-nonalactone; octahydro-2H-chromen-2-one; cis-4-decenal; 3-(3-isopropylphenyl)butanal.

10. A method according to claim 9, wherein the perfume component is selected from at least one of the following: N-ethyl-N-(3-methylphenyl)propionamide; 2-ethyl-N-methyl-N-(3-methylphenyl)butanamide; (4-isopropylcyclohexyl)methanol; 3-methyl-5-phenylpentan-1-ol; 2,2,2-trichloro-1-phenylethyl acetate; isobornyl acetate; allyl amyl glycolate; acetyl cedrene; tetrahydrogeraniol; citronellal; cuminic aldehyde; cis-jasmone; pine American oil; peppermint (Chinese); 1,3,3-trimethyl-2-norbornanol; gamma-nonalactone; octahydro-2H-chromen-2-one; cis-4-decenal; 3-(3-isopropylphenyl)butanal.

11. A method according to claim 9 or 10, wherein the composition is a perfume composition.

12. A method according to claim 11, wherein the perfume composition comprises at least 30% by weight of at least one of the specified perfume components.
